(19) United States
(12) Plant Patent Application Publication
(10) Pub. No.: US 2008/0235837 P1
(43) Pub. Date: Sep. 25, 2008

(54) KALANCHOE PLANT NAME 'FOREVER MIDI RED'

(75) Inventor: Lyndon W. Drewlow, Lompoc, CA (US)

Correspondence Address: THE WEBB LAW FIRM, P.C., 700 KOPPERS BUILDING, 436 SEVENTH AVENUE, PITTSBURGH, PA, 15219, US

(73) Assignee: The Paul Ecke Ranch, Encinitas, CA (US)

(21) Appl. No.: 11/726,731

(22) Filed: Mar. 22, 2007

(51) Int. Cl. A01H 5/00

(52) U.S. Cl. PLT/341

(57) ABSTRACT

‘Forever Midi Red’ is a new variety of kalanchoe that exhibits rich red flowers with a long blooming period.

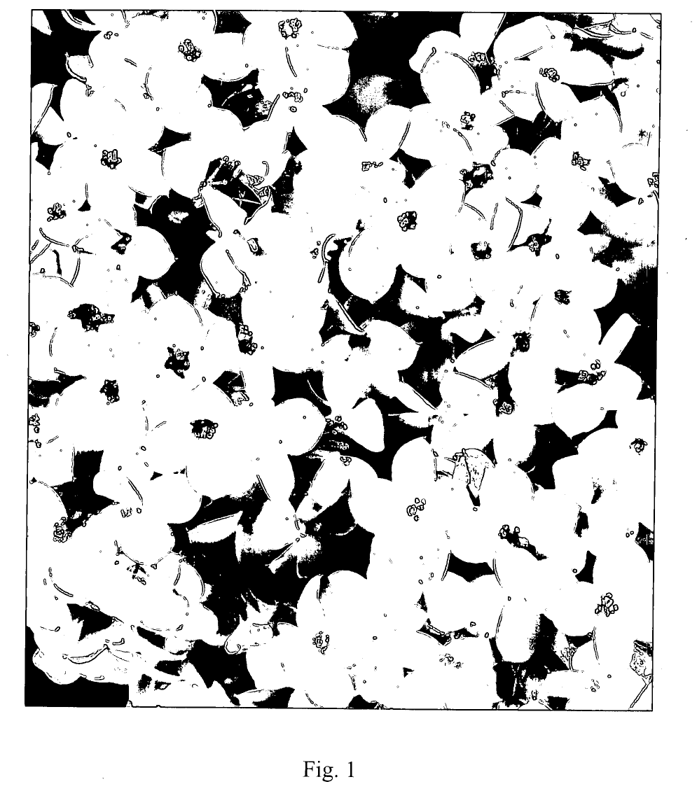

### BOTANICAL CLASSIFICATION

Kalanchoe blossfeldiana

### VARIETAL DENOMINATION

‘Forever Midi Red’

### BACKGROUND OF THE INVENTION

The present invention comprises a new and distinct cultivar of Kalanchoe blossfeldiana known by the varietal name ‘Forever Midi Red’. The new variety was discovered in Lompoc, Calif. The new variety is a result of cross breeding Seedling #98-231-2 (female parent) (unpatented) and Seedling #97-279-2 (male parent) (unpatented). The purpose of the breeding program was to develop a compact, long-lasting and self-branching cultivar. Comparisons between the new variety and its parents cannot be made, as the parental varieties no longer exist. The new variety was first asexually reproduced by tip cuttings in Lompoc, Calif. The new variety has been trial and field tested and has been found to retain its distinctive characteristics and remain true to type through successive propagations. Characteristics that distinguish the new variety from others known to the breeder include:
Mounded and compact growing habit;
Exhibits rich red flowers that do not fade;
Early to flower, 9 to 9 ½ weeks from the start under short days;
Able to be finished in 10 cm pots without growth regulators;
Small leaf size allows for easier sleeving and shipping;
Does not heat delay in night temperatures above 22° C.;
Long blooming with indeterminant inflorescence;
Highly self-branched without pinching; and
Resistant to checking of the red flowers.

The new variety was grown in a 10 cm. container in Lompoc, Calif. in a greenhouse under Dynaglass with 30% shade and 3500 to 4000 foot candles. The new variety was grown under natural, short days having temperatures of 20-22° C. and night temperatures of 16-18° C. The new variety initiates roots in 14 days at 21° C. The time to produce a rooted plant is 21 days at 21° C. Its rooting habit is dense and freely branching with fine and fibrous roots.

Further, the following Tables illustrate differences between the new variety and Kalanchoe varieties ‘Aroi’ (unpatented) and ‘Molly’ (U.S. Plant Pat. No. 12,307), respectively:

TABLE 1

| Characteristics | ‘Forever Midi Red’ | ‘Aroi’ |
|---|---|---|
| Flower color | 45B | 42A |
| Stigma color | White with reddish tint | White |
| Leaf petiole length | 12 mm | 8 mm |
| Flower bud color | 44C near tip | 39A near tip |
| | 144A near calyx end | 146B near calyx |
| Petal size | 8 mm long by | 6 mm long by |
| | 6 mm wide | 5 mm wide |

TABLE 2

| Characteristics | ‘Forever Midi Red’ | ‘Molly’ |
|---|---|---|
| Flower color | 45B | Between 45B and 57B |
| Growth habit | Mounded | Upright |
| Stigma color | White with reddish tint | White |
| Plant height | 14 cm | 18 cm |
| Petal size | 8 mm long by 6 mm wide | 6 mm long by 5 mm wide |

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Lompoc, Calif. The color readings were taken indoors under 3500-4000 foot candles of light. The new variety was twelve weeks from the start of short days when described. Color references are primarily to the 1966 R.H.S. Colour Chart of The Royal Horticultural Society of London.
Plant:
   Form.—Mounded.
   Height (from soil to top of flowers).—14 cm.
   Plant diameter.—20-22 cm.
   Vigor.—Vigorous.
   Branching habit.—Highly self-branching.
   Main stem.—Color: 146B Texture: Glabrous Length: 10 cm Diameter: 1 cm Internode length: 12 mm Strength: Strong.
   Lateral branches.—Color: 146B Texture: Glabrous Length: Basal: 8 to 9 cm Upper: 7-8 cm Diameter: 3 mm Internode length: 8-10 mm Strength: Strong Quantity: 8-10.
   Foliage.—Arrangement: Opposite Number of leaves per stem: 2 Size of leaf: Length: 8-9 cm Width: 5-5.5 cm Shape of leaf (generally): Ovate Shape of apex: Acute to ovate Shape of base: Acute to truncate Texture: Glabrous, coriaceous and succulent Aspect: 45° angle to stem Margin type: Crenate, moderately lobed, shallow cut Fragrance: none Color: Young leaves: Upper surface: 146A Lower surface: 146B Mature leaves: Upper surface: 147A Lower surface: 147B Petiole: Length: 12 mm Diameter: 6 mm Color: 147A Texture: Glabrous Veins: Venation pattern: Pinnate Color: Upper surface: 147A Lower surface: 147B.
Flower:
   Natural flowering season.—January in Lompoc, Calif. — Short days start around October 15^{th}.
   Flower type and habit.—Single, alternate, cyme inflorescense.
   Number of flowers per plant.—250 or more.
   Time of day of flower opening.—Morning, but it takes 3-5 days to fully open.
   Fragrance.—None.
   Longevity.—Up to 2 weeks, depending on environment.
   Flower height.—12 mm, including corolla tube.
   Flower diameter.—15 mm.
   Bud (described just before opening).—Shape: Oblong Diameter: 3 mm Length: 12 mm Color: 44C near tip, 144A near calyx.
   Corolla.—Diameter: 4 mm Depth: 16 mm Tube: Length: 10 mm Diameter: 4 mm.
   Petals.—Arrangement: Whorl of 4 fused at base Quantity: 4 Length: 8 mm Width: 6 mm Shape: Ovate to round Apex: Cuspidate Margin: Entire Texture: Smooth, soft Color: Upper surface: 45B Lower surface: 43C.
   Sepals.—Quantity: 4 Shape: Lanceolate Length: 8 mm Width: 2 mm Color: 146C.
   Main stem peduncles.—Length: 18 mm Diameter: 3 mm Color: 146B Texture: Glabrous Orientation: 45° angle Strength: Strong.
   Pedicels.—Length: 3 mm Width: 3 mm Color: 146B.
   Disease resistance.—Present.
   Seed production.—None observed.
   Fruit production.—None observed.
Reproductive organs:
   Stamens.—Number (per flower): 8 Filament length: 6 mm and approximately 3 mm fused to corolla tube Anthers: Shape: Flat, elliptical Length: Less than 1 mm Color: Yellow-Green Pollen: Color: Yellow Amount: Abundant.
   Pistils.—Length: 10 mm Styles: Length: 3 mm Color: Light yellow Stigma: Shape: Flat Color: White with a reddish tint when mature Ovary color: 146C.

I claim:

1. A new and distinct variety of kalanchoe plant named ‘Forever Midi Red’ as is herein described and illustrated.
